(19) United States
(12) Patent Application Publication
(10) Pub. No.: US 2022/0088538 A1
(43) Pub. Date: Mar. 24, 2022

(54) HOLLOW FIBER FILTRATION SYSTEMS AND METHODS

(71) Applicant: Repligen Corporation, Waltham, MA (US)

(72) Inventor: Rudolf Pavlik, Dover, MA (US)

(73) Assignee: Repligen Corporation, Waltham, MA (US)

(21) Appl. No.: 17/417,618

(22) PCT Filed: Jan. 10, 2020
(86) PCT No.: PCT/US2020/013087

§ 371 Date: Jun. 23, 2021

Related U.S. Application Data
(60) Provisional application No. 62/790,808, filed on Jan. 10, 2019.

(51) Int. Cl. B01D 61/20; B01D 61/18; B01D 63/02; C12M 1/26; C12M 1/00; F04B 45/04; F04B 53/16

(52) U.S. Cl. CPC B01D 61/20; B01D 61/18; B01D 63/02; C12M 33/14; B01D 2315/10; F04B 45/04; F04B 53/16; B01D 2313/243; C12M 47/02

(57) ABSTRACT

The present disclosure provides apparatuses and methods that integrate sensors into bioprocessing systems and, advantageously, do not require significant re-engineering of these systems. The disclosure describes an assembly for alternating tangential flow filtration. The assembly also includes a sensor inserted into the first hemisphere of the pump housing via the port. In various embodiments, the port comprises a tri-clover connector, and the sensor is optionally inserted into the port through a plug sized to substantially occlude the port and to place the sensor in contact with a fluid in the pump housing.

### PRIORITY TO RELATED APPLICATIONS

This application claims priority to U.S. Provisional Application No. 62/790,808, filed on Jan. 10, 2019, and titled “HOLLOW FIBER FILTRATION SYSTEMS AND METHODS.” The foregoing application is incorporated by reference herein in its entirety and for all purposes.

### FIELD OF THE DISCLOSURE

The present disclosure relates generally to filtration systems, and more particularly to an alternating tangential flow filtration unit that includes a sensor, e.g. for sensing viable cells.

### BACKGROUND

Filtration is typically performed to separate, clarify, modify, and/or concentrate a fluid solution, mixture, or suspension. In the biotechnology, pharmaceutical, and medical industries, filtration is vital for the successful production, processing, and analysis of drugs, diagnostics, and chemicals as well as many other products. As examples, filtration may be used to sterilize fluids and to clarify a complex suspension into a filtered “clear” fraction and an unfiltered fraction. Similarly, constituents in a suspension may be concentrated by removing or “filtering out” the suspending medium. Further, with appropriate selection of filter material, filter pore size and/or other filter variables, many other specialized uses have been developed. These uses may involve selective isolation of constituents from various sources, including cultures of microorganisms, blood, as well as other fluids that may be solutions, mixtures, or suspensions.

Biologics manufacturing processes have advanced through substantial process intensification. Eukaryotic and microbial cell cultures to produce recombinant proteins, virus-like particles (VLP), gene therapy particles, and vaccines all now include cell growth techniques that can achieve 10⁷ cells/ml or higher. At these cell densities, it becomes critical to efficiently remove metabolic waste products and refresh the culture with additional nutrients. This is achieved in some bioreactor systems, referred to as “perfusion systems,” by alternating tangential flow hollow fiber filtration (ATF). In these systems, a hollow fiber filter is placed in fluid communication with the bioreactor and fluid flows through the filter are driven by an alternating pump, such as an alternating diaphragm pump. The system may be controlled by a controller, which may operate the pump based on a preprogrammed sequence or in response to a signal from one or more sensors.

More generally, it may be useful or desirable to use sensors in ATF systems to monitor and/or control processes therewithin. Sensors that may be useful include temperature sensors, pH sensors, O₂ partial pressure sensors (O2P), and frequency impedance-based biomass sensors. However, despite the desirability of incorporation of sensors into these systems, there remains a need for fluid handling systems and methods which integrate these sensors.

### SUMMARY OF THE DISCLOSURE

The present disclosure provides apparatuses and methods that integrate sensors into bioprocessing systems and, advantageously, do not require significant re-engineering of these systems. In one aspect, the disclosure relates to a fluid filtration assembly comprising a filter housing having a first end for fluid connection with a fluid storage vessel, a filter cartridge that can be disposed within the filter housing, and a pump assembly coupled at a second end of the filter housing, wherein the assembly includes a pump housing that defines first and second hemispheres such that the first hemisphere is fluidly connected to the filter housing and comprises a port. The pump housing includes a flexible diaphragm fixed between the first and second hemispheres. The assembly also includes a sensor inserted into the first hemisphere of the pump housing via the port. In various embodiments, the port comprises a tri-clover connector, and the sensor is optionally inserted into the port through a plug sized to substantially occlude the port and to place the sensor in contact with a fluid in the pump housing. Alternatively, or additionally, the sensor may be inserted into the port and bonded to the first hemisphere of the pump housing. In some instances, the diaphragm is moveable between first and second positions, e.g., by a mechanical actuator such as a piston, or by the application of positive or negative pressure to the second hemisphere of the pump housing. The first and second hemispheres of the pump housing may, in some cases, comprise radially extending flanges oriented such that opposing face surfaces of the radially extending flanges contact each other when they are clamped together.

In another aspect, the disclosure relates to a bioprocessing fluid handling assembly which includes a cylindrical fixture comprising an aperture and a sensor disposed within the aperture and secured to the fixture. The sensor may be secured to the fixture by bonding, or if the aperture comprises a tri-clover port, the sensor may be secured to the fixture by a plug inserted within the aperture which is clamped within the tri clover port when it is closed.

Additional aspects and embodiments will be evident to skilled artisans in view of the disclosure that follows.

### BRIEF DESCRIPTION OF THE DRAWINGS

The accompanying drawings illustrate preferred embodiments of the disclosed method so far devised for the practical application of the principles thereof, and in which:

FIG. 1a and FIG. 1b are schematic views of an alternating tangential flow filtration system according to the disclosure;

FIG. 2 is a side view of a pump and filter assembly for use with the system of FIG. 1;

FIG. 3 is a cross-section view of the pump and filter assembly of FIG. 2 taken along line 3-3 of FIG. 2;

FIG. 4 is a partial detail view of the pump and filter assembly shown in FIG. 3;

FIG. 5 is an isometric view of an alternative embodiment of a pump and filter assembly for use with the system of FIG. 1;

FIG. 6 is a side view of a pump and filter sensor assembly according to certain embodiments of this disclosure;

FIG. 7 is a cross-section view of the pump and filter sensor assembly depicted in FIG. 6;

FIG. 8 is a cross-section view of a sensor assembly fitting according to certain embodiments of this disclosure;

FIG. 9 is a side view of the sensor assembly fitting depicted in FIG. 8;

FIG. 10 is a cross-section view of a sensor assembly fitting according to certain embodiments of this disclosure;

FIG. 11 is a side view of the sensor assembly fitting depicted in FIG. 10; and

FIGS. 12a and 12b show cross-sectional assembled (12a) and exploded (12b) views of a sensor assembly according to certain embodiments of this disclosure.

### DETAILED DESCRIPTION

Alternating Tangential Flow Filters

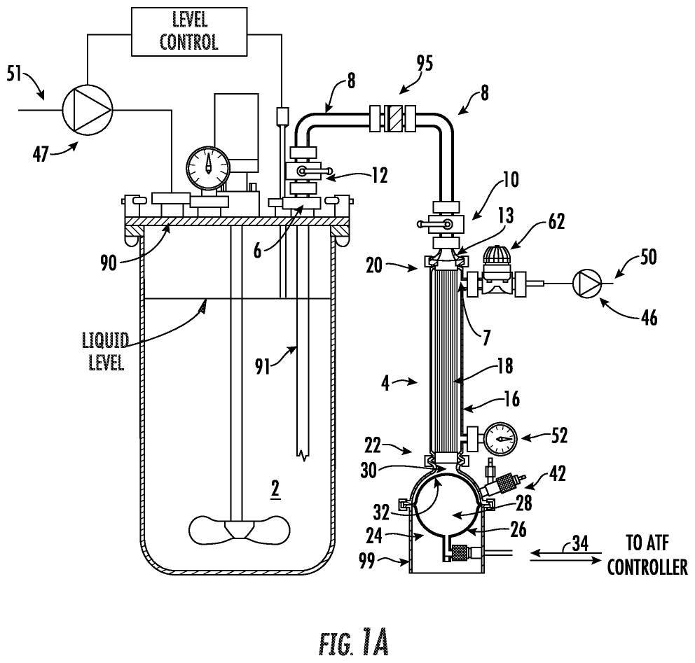

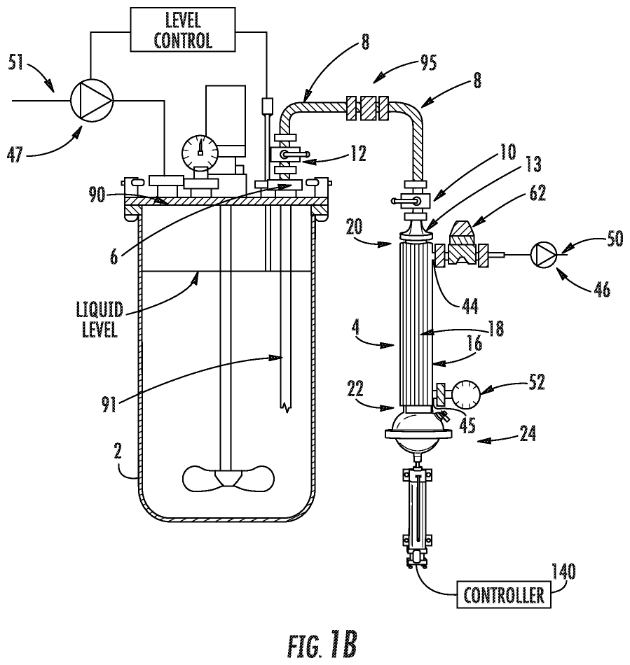

Referring to FIGS. 1a and b, in certain fluid filtration systems according to this disclosure a process vessel 2 is connected via a fluid connector to a filter containing compartment 4. The vessel 2 may be any suitable container for a fluid to be filtered. For example, it may be a bioreactor, a fermentor or any other vessel, nonexclusively including vats, barrels, tanks, bottles, flasks, containers, and the like which can contain liquids. The vessel may be composed of any suitable material such as plastic, metal such as stainless steel, glass, or the like. The fluid connector serves to direct a fluid from the storage vessel into an entrance end of a filter containing compartment.

The fluid connector may comprise a vessel port 6, suitable for flowing fluid into and out of the vessel, attached to joint 8 which is in turn is connected to the entrance end of the filter containing compartment 4. Suitable ports nonexclusively include any sanitary, leak-proof fittings known in the art such as a compression, standard Ingold or a sanitary type fitting. Suitable joints nonexclusively include pipes, tubes, hoses, hollow joint assemblies, and the like. For a penetration into a lower side of a vessel, the most preferred fluid connecting means is an L-shaped pipe as shown. The joint may vary from one system to another, based on the configuration and requirements of the vessel and process. Joint 8 is connected both to the vessel port 6 and the entrance end of the filter containing compartment 4 via appropriate valves 10 and 12. The joint 8 may be attached to the valves 10 and 12 by suitable clamps, such as a triclamp sanitary fitting or the like. This does not preclude the use of other appropriate connections. The filter containing compartment 4 comprises a filter housing 16 which holds a replaceable filter element cartridge 18. The connection between valve 10 and housing 16 may be direct or indirect should there be a mismatch between the corresponding fittings. In one example, should the housing 16 contain a 2.5 inch sanitary end and valve 10 contain a ½ sanitary end, a 2.5 by ½ inch sanitary adapter 13 may be used to join the two ends.

The filter containing compartment 4 preferably has an entrance end 20 and an exit end 22. Entrance end 20 is attached directly to valve 10, while exit end 22 is connected to a diaphragm pump 24, e.g., by means of clamp. Suitable materials for the housing of the filter containing compartment nonexclusively include plastic, metal, such as stainless steel, glass, and the like. Suitable removable filter elements nonexclusively include hollow fiber filters, screen filters, and the like. The filter element can optionally be removed from the fluid filtration system before, during, or after the filtration process. Suitable hollow fiber filtration membranes or screen filters are commonly available from various vendors.

A diaphragm pump 24 is used to move the fluid from the vessel 2 through the filter 18 in the filter containing compartment 4 into the pump 24 and then reversing the fluid flow from pump 24 back through the filter to vessel 2. In this way, an alternating tangential flow of fluid is generated through filter 18. In the case where filter 18 is a hollow fiber cartridge, both ends, the entrance end 20 and the exit end 22, of 18 are sealed against the housing wall to prevent mixing of the retentate and filtrate.

The diaphragm pump 24 comprises a pump housing 26 separated into first and second interior chambers 28 and 30 by an internal diaphragm 32. The diaphragm is flexible and is preferably fixed inside the housing via a leak proof, sanitary fitting. The diaphragm may be uniform in thickness, or may vary somewhat in thickness or shape, as the process may require. In one example, a thicker region is formed at the center of the diaphragm. The thicker region may face towards compartment 28. During an exhaust cycle or during sterilization, when the diaphragm is forced into exhaust/air inlet port, such a thicker region will offer the diaphragm added structural support.

In some instances, as shown in FIG. 1a, the diaphragm pump may be pneumatically activated. The diaphragm pump has an entrance end through which fluid flows from the exit end 22 of the filter containing compartment 4 to the second, interior chamber 30 of pump 24. Pump chamber 28 isolates and contains the mechanism for driving the diaphragm within pump 24 without contaminating the fluid content in the adjacent chamber 30. The pump is pneumatically actuated by alternately feeding a gas, such as air through a reversible inlet/exhaust line 34. The inlet/exhaust line 34 is attached to pump 24 via a connector such that when the gas is passed through the line 34, it is injected into the first chamber 28 of the pump and fills the first chamber 28 with the gas expanding the chamber and flushing any fluid in the second chamber 30 in a direction toward and through the filter 18. Typically, but not exclusively, a controlled addition of compressed air into through 34 may be used to expand chamber 28, inversely, reducing the volume in the adjacent pump chamber 30, driving the content from chamber 30 to vessel 2. When the gas is drawn back through line 34, such as by a vacuum source, not shown, in the indicated exhaust direction, the diaphragm 32 is drawn towards the gas inlet. Chamber 28 decreases in volume, allowing flow from vessel 2 through the filter module 18 and into expanding chamber 30. Bidirectional flow control of air through line 34 may be regulated by microprocessor control of a suitable 3-way or 4-way solenoid valve, not shown. This action repeats drawing fluid back and forth from the vessel 2, through the filter, and chamber 30 causing an alternating flow tangentially through the filter 18. Chamber 28, which is connected to a gas inlet/exhaust line 34 may contain a hydrophobic filter for allowing a gas such as air to freely flow through line 34 while preventing liquid flow therethrough. The fluid filtration system preferably also comprises a controller for controlling the movement of the diaphragm within the pump housing. FIGS. 1A-B show an alternating tangential flow controller. The controller may comprise a pressure measuring device such as a pressure transducer which serves to monitor and or regulate the pressure in chamber 28 and 30 relative to the process vessel 2. It may be used to trigger a reversal of gas flow via line 34 into and out of chamber 28 and hence fluid flow into and out of chamber 30 by triggering the switching and controlling the expansion and contraction of the diaphragm within the pump housing. Other means of switching the movement of the diaphragm, such as the use of proximity switches, are also within the contemplation of the invention. It is noted that pump chambers 28 and 30 need not be of the same size nor do they have to be spherical as shown. They may be adjusted to the requirement of the process by the alternating tangential flow (ATF) controller as shown. As a result, fluid flow back and forth through the filter is controlled. For example, when working with animal cells, cells may be damaged if chamber 28 expands to the point where the diaphragm 32 is forced against the inner pump wall of chamber 30. To minimize or prevent the entrapment of cells between the pump wall and diaphragm, the chamber 30 wall may have a somewhat larger radius than the radius of the chamber 28 wall. With the diaphragm 32 having the same radius as chamber 28, expansion of chamber 28 need not drive the diaphragm to the chamber 30 wall, and sufficient space is maintained between the diaphragm and the pump wall. Controlled expansion of chamber 28, the selection of diaphragm materials and, if desired, the use of sensors may accurately control the position of the diaphragm in the pump.

The diaphragm pump can also be mechanically, rather than pneumatically, actuated. In one exemplary arrangement, the plunger activation system can include a controller and a two-directional movement device such as a servo motor, cam, pneumatic or electrical actuator. It will be appreciated that the plunger activation system, such as servo motor, cam, pneumatic or electrical actuator, will be connected to the plunger pump via a connection/disconnection coupling, and will be applied at the point of use. Thus, the plunger activation system will not be sterilized.

The plunger pump has a liquid side which is sealed to the filter housing hemisphere and an external side which has a mechanical connection coupling positioned symmetrically in the center of a plunger. The plunger material can be a rubbery, medical grade thermoplastic material, a silicone or other appropriate elastomeric material. The plunger movement in a “pull” cycle generates suction (i.e., drawing liquid toward, or into, the pump), and in a “push” cycle provides liquid extraction or expulsion (i.e., pushing liquid away from, or out of, the pump). The stroke distance of the plunger pump in the pull and push mode can be predetermined and/or adjusted as appropriate to accommodate the particular size of the filter assembly. In some embodiments the stroke distance can be automatically controlled by a device such as a linear encoder.

The actuation system and the plunger can be universal for all available sizes of filter assemblies. Confirmation of pump size can, in some embodiments, automatically reset the stroke distance to be appropriate for a particular filter assembly size. Since travel distance for each pump size is different, the actuation travel will be set for the largest pump size, which requires the longest stroke. Any other pump size will have a shorter stroke, and thus, the travel will be adjusted based on a specific pump stroke requirement. Pump stroke for the smaller filter assemblies will start from the end “push” position, and distance of the “pull” travel will correspond with the appropriate volume required by the particular filter size.

Speed of movement can be part of a separate setting entered directly into a control module by a user, or it can be dictated by a selectable recipe. One plunger “push” and “pull” motion represents the pump cycle. The number of cycles per minute represents plunger pump flow, which is typically measured in liters per minute. Greater or fewer cycles per minute will provide higher or lower pump flow. The desired pump flow can be entered manually or can be a part of a process recipe.

Referring now to FIG. 1B a fluid filtration system includes a process vessel 2 connected via a fluid connector to a pump and filter assembly 4. The vessel 2 may be any suitable container for housing a fluid to be filtered. For example, it may be a bioreactor, a fermentor or any other vessel, nonexclusively including vats, barrels, tanks, bottles, flasks, containers, and the like which can contain liquids. The process vessel 2 may be composed of any suitable material such as plastic, metal such as stainless steel, glass, or the like. The fluid connector serves to fluidly couple the process vessel 2 to the pump and filter assembly 4.

The fluid connector may comprise a vessel port 6 coupled to the process vessel 2, and an appropriate section of piping 8 which is in turn is connected to the entrance end of the pump and filter assembly 4. The vessel port 6 can be any appropriate sanitary, leak-proof fittings known in the art such as a compression, standard Ingold or a sanitary type fitting. The piping 8 can alternatively include tubes, hoses, hollow joint assemblies, and the like. In addition, the piping 8 can include appropriate valves 10 and 12 for selectively isolating, or allowing, flow between the vessel 2 and the pump and filter assembly 4.

In the illustrated embodiment, a vessel port 6 is provided through a head plate 90 of the process vessel 2. A dip tube 91 is used to connect to the liquid in the process vessel 2 to piping 8. It will be appreciated that piping 8 need not be rigid and flexible connection 95 may facilitate making and breaking connection between the vessel 2 and the filter assembly 4. In some embodiments, filtered harvested liquid can be collected from the filter assembly 4 through line 50. The harvested liquid may be restored by a level control mechanism that activates an additional pump 47 to pump liquid into the vessel through line 51.

The pump and filter assembly 4 includes a filter housing 16 that holds a filter element cartridge 18. The housing 16 can include a fluid harvest port 44 suitable for removing filtered fluid from the housing. A harvest line 50 can be coupled to the fluid harvest port 44, and can include a valve 62 and a filtrate pump 46 for controlling the removal of filtered fluid from the system. Pressure in the housing 16 may be monitored by a pressure valve or transducer 52 coupled to the housing via a monitoring port 45.

The pump and filter assembly 4 can include a filter housing 16 and a plunger pump 24 coupled thereto. The filter housing can have an entrance end 20 and an exit end 22. The entrance end 20 can be attached to the piping 8, for example, via valve 10 and adapter 13. The exit end 22 can be connected to the plunger pump 24 as will be described in greater detail later.

The filter housing 16 can be made from plastic, metal, such as stainless steel, glass, and the like. Suitable removable filter element cartridges 18 (for reusable filter housings) or complete, permanent housings (for single use systems) include hollow fiber filters, screen filters, and the like. In one non-limiting example embodiment, the filter element cartridge 18 is a hollow fiber filter. According to the disclosure, pump and filter assembly 4 can be configured for single use (i.e., disposable), with the filter housing 16, filter cartridge 18 and plunger pump 24 provided together as an integral assembly.

Various advantages exist in providing the pump and filter assembly 4 as a single-use (disposable) assembly. For example, the assembly can be set up with minimal handling and does not require cleaning or sterilization by the user, since the components are supplied sterile and in a form ready to use with minimal preparation and assembly. This can result in cost savings due to reduced labor and handling by the user along with elimination of a long autoclave cycle. Furthermore, at the end of their use, the assembly can be readily discarded without cleaning. A disposable assembly reduces risk of contamination and assembly by operators, and do not require lengthy validation procedures for operation/sterilization. The components of the assembly also can be lighter and easier to transport, and are less expensive and take up less storage space compared to stainless steel or glass units.

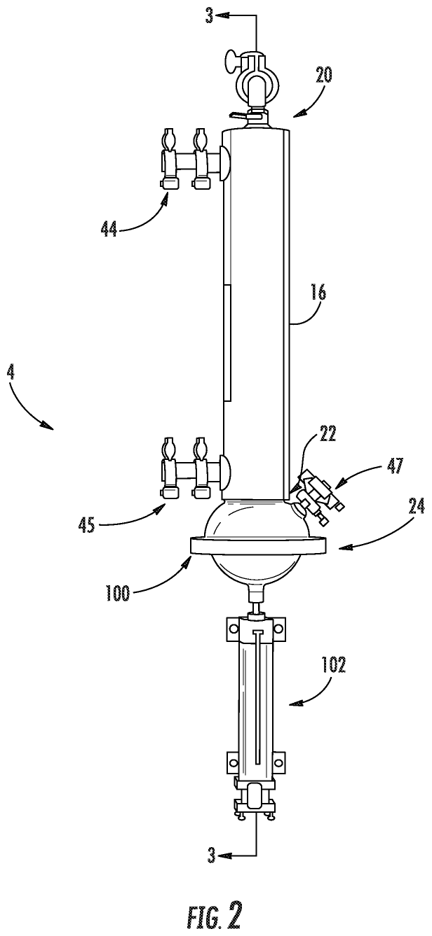

FIG. 2 illustrates an example pump and filter assembly 4 including filter housing 16 enclosing a filter (not shown) and a plunger pump 24. In one non-limiting example embodiment, the pump and filter assembly 4 is a single use integral assembly for filtering fluid stored in the process vessel. The pump and filter assembly 4 can include an entrance end 20, an exit end 22, a fluid harvest port 44, and a monitoring port 45 for coupling a pressure valve or transducer as previously mentioned. A sample port can be provided for coupling a sampler valve (not shown) to the filter housing 16.

A sampler valve may be used for a variety of purposes including sampling the quality of the fluid in the plunger pump 24, injecting or expelling liquid or gas into and out of the pump, and injecting sterilizing steam into the system and/or removing resulting steam condensate from the system. For example, the sampler valve may be suitable for injecting air into the system to expel liquid from the system into the process vessel prior to detachment of the filter system from the process vessel; conversely, it may be used to purge air from the system prior to initiating alternating tangential flow.

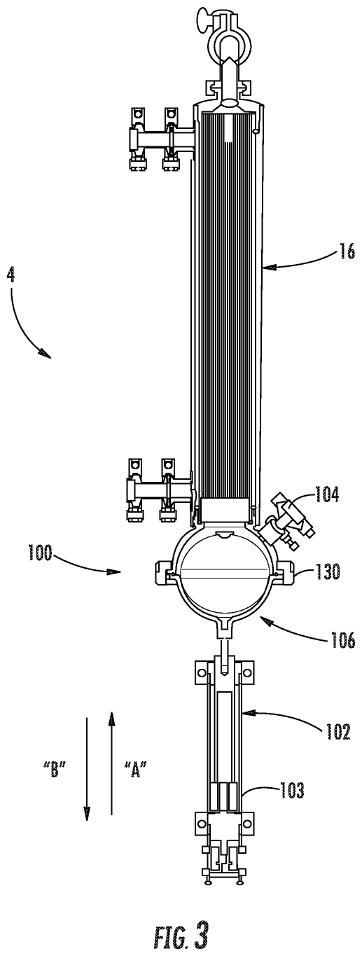

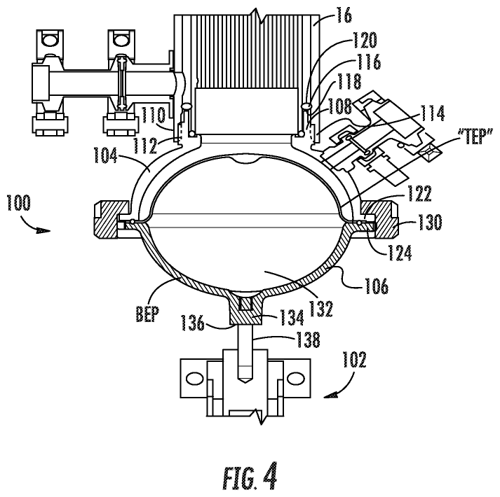

The plunger pump 24 can include a housing portion 100 and an actuator portion 102. As shown in greater detail in FIGS. 3 and 4, the housing portion 100 may include a rigid portion 104 and a flexible portion 106 coupled together. The flexible portion 106 may also be coupled to the actuator portion 102 so that the flexible portion 106 is movable with respect to the rigid portion 104 in response to activation of the actuator portion. The actuator portion 102 may include a cylinder housing 103, and a driven rod portion 138 that is selectively movable within the cylinder housing. As will be described in greater detail below, a servo motor, cam, pneumatic or electrical actuator can be used to selectively move the rod portion 138 in the directions of arrows “A” and “B” to cause the plunger pump to move fluid through the filter housing 16 in a desired manner.

As can be seen in FIGS. 3 and 4, an upper end 108 of the rigid portion 104 of the housing portion 100 is coupled to a lower end 110 of the filter housing 16 in a manner that allows fluid to flow freely therebetween. In the illustrated embodiment, the upper end 108 of the rigid portion includes external threads 112 sized and configured to mate with internal threads 114 of the lower end 110 of the filter housing 16. An O-ring 116 is disposed between an upper end surface 118 of the rigid portion 104 and a lower end surface 120 of the filter housing 16 to provide fluid-tight engagement between the two. It will be appreciated that although a threaded connection is disclosed, other coupling and sealing arrangements can be used without departing from the spirit of the disclosure. In addition, it is contemplated that the rigid portion 104 of the housing portion 100 can be formed as an integral part of the filter housing 16.

As best seen in FIG. 4, the rigid portion 104 and flexible portion 106 of the housing portion 100 can each be bell-shaped members that can be coupled together to provide the housing portion with a globe shape having an interior volume 132 defined by respective inner surfaces of the rigid and flexible portions. The rigid portion 104 and flexible portion 106 have respective radially extending flanges 122, 124. The combination of the flanges 122 and 124, corresponding “O” ring like feature of the flexible portion 106 of the plunger, and the durometer of the flexible portion guarantee an integral connection secured by a clamp (referred to as a “nut”) 130. In some embodiments, the flexible portion 106 can be formed from an elastomer that is overmolded on the rigid portion 104, thus eliminating the need for a clamp portion.

The flexible portion 106 may have a bell, accordion or bellows shape. As will be appreciated, expansion or contraction of the flexible portion 106 can generate vacuum and pressure required to initiate movement of fluid between the plunger pump 24 and the process vessel 2. Friction between the internal plunger surfaces (liquid contact area) can be mitigated by the plunger shape design. For example, when the flexible portion 106 moves to each end position of the stroke (i.e., the bottom end position “BEP” and the top end position “TEP” explained below), the internal surfaces of the plunger will not be in contact.

The flexible portion 106 may include a plunger-engaging portion 134 disposed in or on a bottom surface thereof. The plunger-engaging portion 134 may include a recess 136 for receiving the rod portion 138 of the actuator portion 102. In the illustrated embodiment, the plunger-engaging portion 134 is aligned with a center of the filter housing 16. Thus arranged, movement of the actuator portion 102 in the direction of arrow “A” causes an even deformation of the flexible portion 106 with respect to the rigid portion 104.

As the actuator portion 102 drives the rod portion 138 in the direction of arrow “A” (i.e., toward the rigid portion 104), the flexible portion 106 deforms and moves toward the rigid portion. In one embodiment, the rod portion 138 is driven to move the flexible portion 106 from a bottom end position “BEP” to a top end position “TEP”. As will be appreciated, when the flexible portion 106 is in the BEP, the interior volume 132 of the housing portion 100 is a first value, and when the flexible portion is in the TEP the interior volume of the housing portion 100 is a second value, where the second value is less than the first value. Thus, as the rod portion 138 moves the flexible portion 106 from the BEP to the TEP (i.e., the direction of arrow “A”), the flexible portion forces liquid contained in the housing portion 100 up into the filter housing 16 and back into the vessel 2. In reverse, when the rod portion 138 moves the flexible portion 106 from the TEP to the BEP (i.e., in the direction of arrow “B”), liquid is drawn from the vessel 2, through the filter cartridge 18 in the filter housing 16, and into the housing portion 100.

As seen in FIGS. 4 and 5, in some embodiments, the actuator portion 102, and thus operation of the plunger pump 24, can be automated via a controller 140. The controller may include an appropriate processor and associated memory (not shown). The processor may execute instructions for actuating the plunger pump 24 according to a desired set of cycle parameters (e.g., stroke distance, stroke rate). The actuator portion 102 can include a linear encoder (not shown) that can monitor the position of the rod portion 138 and provide associated position information to the processor and/or other component of the controller 140. The location of the rod portion 138 can be monitored throughout an entire actuation cycle of the plunger pump 24. Thus, the controller 140 can monitor the end positions (BEP and TEP) of the flexible portion 106, and can use this information to determine and/or control fluid volume displacement over a particular time period. Due to the mechanical engagement between the rod portion 138 and the flexible portion 106 of the pump 24, the stroke distance can be known with a relatively high degree of confidence at any point of the actuation process.

In some embodiments, the stroke distance (i.e., the distance that the rod portion 138 and the flexible portion 106 are moved in a given direction) can be preset by the controller and is dependent upon the size of the particular pump and filter assembly 4 used. Using the linear encoder of the actuator portion 102, the stroke distance can be appropriately controlled and confined to the predetermined value by the controller 140. In this manner, the controller 140 and actuator portion 102 can be used universally for all available sizes of pump and filter assemblies. Using an automatic stroke distance preset for each filter is a convenient way to prevent a short or over stroking the plunger.

Confirmation by the controller 140 of the size of a particular pump and filter assembly may result in an automatic reset of the stroke distance. A stroke speed can be entered or otherwise set in the controller 140 either directly or dictated by a recipe or appropriate algorithm.

The connection between the actuator portion 102 and the controller 140 is illustrated as being hard wired, but it will be appreciated that the two may be wirelessly connected. The controller can have an interaction interface that can allow the user to control stroke distance, stroke movement profile, flow of the plunger pump and control of any ancillary devices related to the functions of the filter assembly.

In use, the plunger pump can generate an alternating tangential flow through the filter cartridge when the flexible portion 106 is driven via the actuator portion 102. The plunger pump can generate a reversible flow of liquid such as a culture suspension, back and forth, between the process vessel and plunger pump. For example, flow from the interior volume 132 of the housing portion 100 through the filter cartridge to the process vessel is generated by moving the flexible portion 106 of the pump in the direction of arrow “A” (i.e., from the BEP to TEP). Movement of flexible portion 106 expels liquid from the interior volume 132 of the housing portion 100, moving the liquid towards the process vessel, and generating a tangential flow in one direction. Final, filtered product is removed through a port, for example, by a peristaltic pump. In the reverse, when the flexible portion 106 of the pump is moved in the direction of arrow “B” (i.e., from TEP to BEP), the pressure in the interior volume 132 of the housing portion 100 is decreased relative to the pressure in vessel. Thus, the flow path is reversed, and liquid flow from the process vessel back to the interior volume 132 of the housing portion 100, generating tangential flow in the opposite direction. Final filtered product is removed through a port by, for example, a peristaltic pump. Flow from pump to the process vessel and return from the process vessel to the pump completes one cycle.

The cycle rate and the flow rate between the plunger pump and the process vessel will depend primarily on the configuration of the pump and the control mechanism used to regulate the cycle.

FIG. 5 shows a single actuator portion 102 used to simultaneously actuate a pair of pump and filter assemblies 4a, 4b, including pumps 24a, 24b, filter housings, and respective filters 18a, 18b. Although the illustrated embodiment shows a single actuator portion 102 for actuating two pump and filter assemblies 4a, 4b, it will be appreciated that a greater number of pump and filter assemblies could be controlled by a single actuator portion 102, which, in turn, can be controlled by a single controller. In this arrangement, a single actuator portion 102 is connected to rigid linkages 103a, 103b which are connected to the flexible portions 106a, 106b of the pumps 24a, 24b via connecting couplings in the manner previously described. Pumping actuation for this arrangement is identical to the actuation described in relation to FIGS. 1-4.

Sensors, Sensor Assemblies and Fittings

In many cell culture processes, it is desirable to monitor certain process variables, such as temperature, pH, optical density or turbidity, and/or viable cell density. The various embodiments of the present disclosure meet this need for continuous tangential flow filtration systems and methods such as those described above. In some cases, existing connection ports on filter and/or pump fittings are used as sensor receptacles during active filtration runs. An adapter may be used to adapt the port for use with a sensor. For instance, as described in greater detail below, one exemplary adapter for a tri-clover connector comprises a “plug” structure that includes a tri-clover flange for clamping and an inner portion that secures the sensor and, optionally, places it into or adjacent to a fluid path within the fitting.

The systems and methods of this disclosure overcome a number of technical challenges. First, they allow existing ports on ATF fittings to be repurposed without compromising the integrity of the port. During ATF runs, fittings must withstand significant pressures or risk contamination and/or structural failure. Accordingly, adapters disclosed herein may utilize flanges similar in size, shape and mechanical properties to tri-clover port gaskets currently used in the art. Second, they do not introduce dead spaces into fluid flows. In some cases, this is accomplished by sizing and shaping the adapter to sit flush with an interior surface of a fitting in which it is deployed.

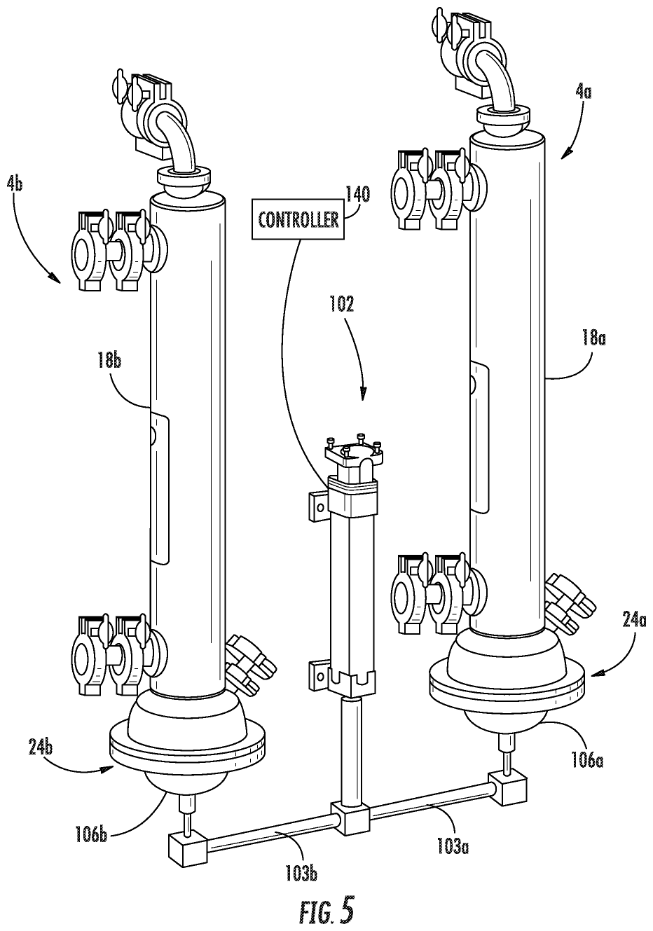

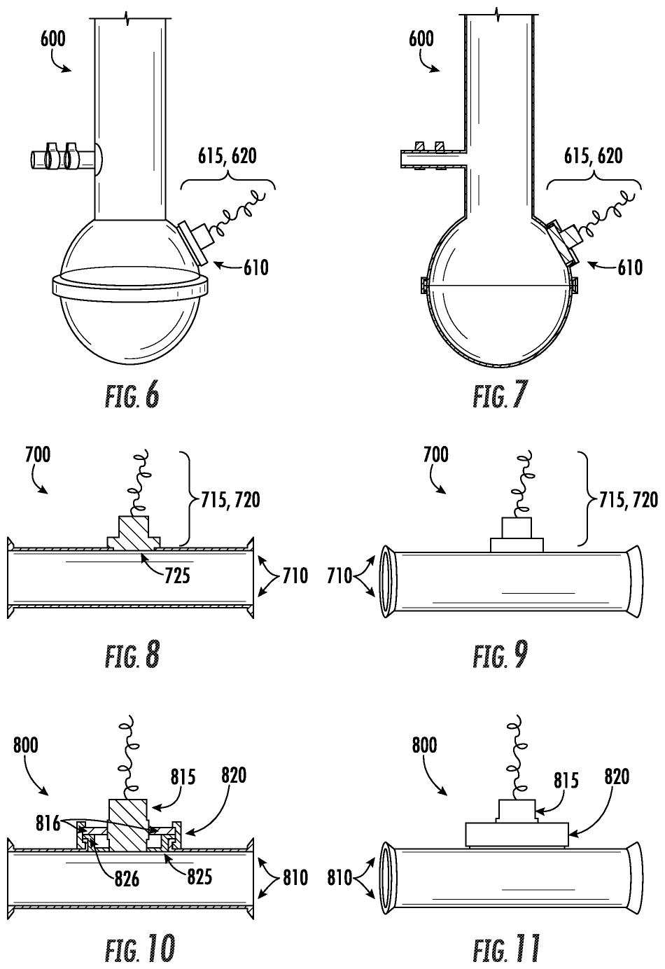

Turning to FIGS. 6 and 7, an alternating diaphragm pump and tangential flow filtration housing 600 includes a port 610, such as a tri-clover port. The port includes a sensor 615 and adapter 620, both of which are described in greater detail below.

FIGS. 8 and 9 depict a linear fitting 700 that includes a sensor 715 and adapter 720. These differ from the adapter 620 described above in their use of a flat interface 725 between the adapter 720 and an aperture or socket in a wall of the fixture 700. The flat interface 725 may comprise any appropriate bonding material, including without limitation a pressure sensitive adhesive, an epoxy or other reactive polymer material, or a mated structure such as male and female threads, etc. The flat interface 725 may also be any suitable shape, e.g., annular or disc shaped, and may be solid or perforated by holes, slots, to place the sensor 715 in fluid contact with a fluid in the fitting etc. The fitting 700 also optionally includes one or more flanged ends 710, that can be mated with another fixture, e.g., by a tri-clover clamp.

Next, FIGS. 10 and 11, depict a linear fitting 800 that differs from the fitting described above in their use of an adapter or plug 825 for a tri-clover port 820. The adapter or plug 825 comprises a flange 826 that mates with an optional gasket 816. The fitting also optionally includes one or more flanged ends 810, as described above.

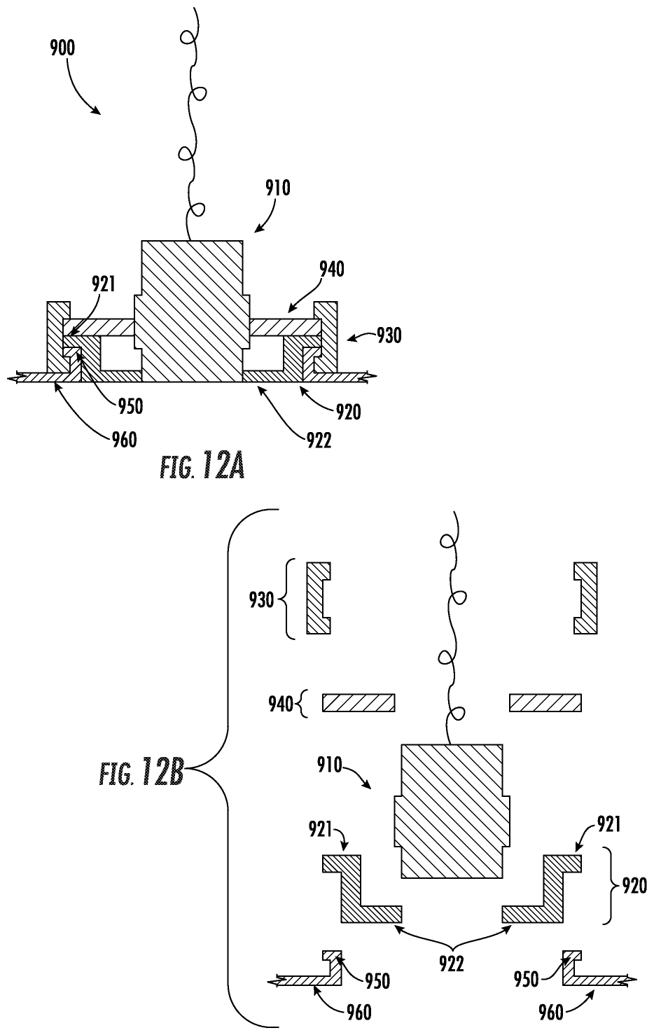

FIGS. 12a and 12b show assembled and exploded cross-sectional views of tri-clover port 900 into which a sensor 910 and adapter 920 are deployed. The tri-clover port 900 includes a clamp 930, a sealing cap 940 comprising a flange sized and shaped to fit within the clamp 930 when the port 900 is sealed. The sealing cap 940 also includes an aperture sized to fit the sensor 910 and, optionally, to secure the sensor 910 by means, e.g., of an interference fit. To accomplish securing of the sensor 910, the sealing cap 940 may include a chamfer or flange configured to mate with a corresponding portion of the sensor 910. The adapter 920 is a plug structure that includes a flange 921 that mates with the flanged portion of the sealing cap 940 and a fitting flange 950 when sealed using the clamp 930. The adapter 920 also includes a flush surface 922 that is flush with an interior surface 960 of the fixture comprising the port 900. The flush surface 922 optionally includes an aperture, perforation, slot, etc. to allow the sensor to contact the fluid in the fixture; to prevent the insertion of the sensor 910, too far into the fixture, the adapter 920 optionally includes a flange, chamfer or other structure.

### CONCLUSION

While the present disclosure has focused on a handful of exemplary embodiments, numerous modifications, alterations and changes to the described embodiments are possible without departing from the spirit and scope of the disclosed arrangement, as defined in the appended claims. Accordingly, it is intended that the present arrangement not be limited to the described embodiments, but that it has the full scope defined by the language of the following claims, and equivalents thereof.

1. A fluid filtration assembly, comprising:
a filter housing having a first end for fluid connection with a fluid storage vessel;
a filter cartridge disposable within the filter housing;
a pump assembly coupled at a second end of the filter housing, the pump assembly comprising:
a pump housing defining first and second hemispheres, the first hemisphere fluidly connected to the filter housing and comprising a port; and
a diaphragm fixed in the pump housing between the first and second hemispheres; and
a sensor inserted into the first hemisphere of the pump housing via the port.

2. The fluid filtration assembly of claim 1, wherein the port comprises a tri-clover connector.

3. The fluid filtration assembly of claim 2, wherein the sensor is inserted into the port through a plug sized to substantially occlude the port and to place the sensor in contact with a fluid in the pump housing.

4. The fluid filtration assembly of claim 1, wherein the sensor is inserted into the port and bonded to the first hemisphere of the pump housing.

5. The fluid filtration assembly of claim 1, wherein the diaphragm is moveable between first and second positions.

6. The fluid filtration assembly of claim 5, wherein the diaphragm is moved between the first and second positions by a change in a pressure of the second hemisphere,

7. The fluid filtration assembly of claim 5, wherein the diaphragm is moved between the first and second positions by a mechanical actuator.

8. The fluid filtration assembly of claim 1, wherein the first and second hemispheres of the pump housing each comprise radially extending flanges, such that opposing face surfaces of the radially extending flanges contact each other when clamped together.

9. A bioprocessing fluid handling assembly, comprising:
a cylindrical fixture comprising an aperture; and
a sensor disposed within the aperture and secured to the fixture.

10. The bioprocessing fluid handling assembly of claim 9, wherein the sensor is secured to the fixture by bonding.

11. The bioprocessing fluid handling assembly of claim 9, wherein the aperture comprises a tri-clover port, and the sensor is secured to the fixture by a plug inserted within the aperture.
